(19) United States
(12) Patent Application Publication
(10) Pub. No.: US 2023/0151348 A1
(43) Pub. Date: May 18, 2023

(54) APPLICATION OF MAL33 GENE DELETION IN IMPROVING TOLERANCE OF SACCHAROMYCES CEREVISIAE TO INHIBITORS IN THE LIGNOCELLULOSE HYDROLYZATES

(71) Applicant: Qilu University of Technology (Shandong Academy of Sciences), Jinan (CN)

(72) Inventors: Lili XU, Jinan (CN); Xiaoming BAO, Jinan (CN); Yun WANG, Jinan (CN); Tianqing XIA, Jinan (CN); Chenhao LI, Jinan (CN); Fangqing WEI, Jinan (CN); Ai YUAN, Jinan (CN)

(73) Assignee: Qilu University of Technology (Shandong Academy of Sciences), Jinan (CN)

(21) Appl. No.: 18/045,178

(22) Filed: Oct. 10, 2022

(30) Foreign Application Priority Data
Nov. 17, 2021 (CN) 202111361848.3

(51) Int. Cl. C12N 15/01; C12N 9/22

(52) U.S. Cl. CPC C12N 15/01; C12N 9/22; C12N 2500/32; C12N 2500/34; C12N 2500/74

(57) ABSTRACT

An application of MAL33 gene deletion in improving the tolerance of Saccharomyces cerevisiae to inhibitors in a lignocellulose hydrolyzate is provided. The tolerance of the present MAL33 gene-deleted Saccharomyces cerevisiae strain to acetic acid is greatly improved, and the tolerance of the Saccharomyces cerevisiae strain to other typical inhibitors and H₂O₂ in the lignocellulose hydrolyzate is also improved. The lag period of the Saccharomyces cerevisiae strain in a glucose and xylose medium (YPDX) with 3.5 g/L acetic acid is shortened by 24 h. The fermentation period of the Saccharomyces cerevisiae strain to produce ethanol through co-utilization of glucose and xylose is shortened by 20 h. The growth of the Saccharomyces cerevisiae strain in a glucose and xylose medium (YPDX) with a mixed inhibitor and the ethanol production of the Saccharomyces cerevisiae strain through the co-fermentation of glucose and xylose are superior to those of a control strain.

### CROSS REFERENCE TO THE RELATED APPLICATIONS

This application is based upon and claims priority to Chinese Patent Application No: 202111361848,3, filed Nov. 17, 2021, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBQCJN-RLH051-Sequence-Listing.xml, created on 10 Oct. 2022, and is 11,355 bytes in size.

### TECHNICAL FIELD

The present disclosure relates to an application of MAL33 gene deletion in improving the tolerance of Saccharomyces cerevisiae to inhibitors in the lignocellulose hydrolyzate and belongs to the field of bioengineering.

### BACKGROUND

In recent years, second-generation ethanol fuel produced from abundant and cheap lignocellulose raw materials (such as agricultural and forestry wastes) is expected to replace non-renewable fossil energy, which can alleviate the current global energy crisis and reduce environmental pollution. Second-generation ethanol fuel has received extensive attention worldwide. When lignocellulose raw materials undergo pretreatment, enzymolysis, and the like, carbohydrates such as pentoses and hexoses that can be utilized by microorganisms, such as Saccharomyces cerevisiae, are released, and various inhibitors including weak acids, furans, and phenol compounds are also produced.

Weak acids mainly include formic acid, acetic acid, and levulinic acid. As a typical inhibitor in the lignocellulose hydrolyzates, acetic acid has the highest content therein. The concentration of acetic acid is generally between 1 g/L to 10 g/L, which is related to the pretreatment process. Acetic acid has the following adverse effects on cells: (1) Acetic acid causes DNA damage, resulting in a decrease in DNA and RNA synthesis rates and metabolic activity. (2) To maintain a normal growth environment, sufficient energy needs to be released through ATP hydrolysis to drive a proton pump, thereby expelling a large amount of H⁺ accumulated in cells. The above process consumes a large amount of ATP, such that the supply of ATP for cellular metabolism will be insufficient, which results in reduced cell viability and ultimately affects the fermentation process of microorganisms. (3) Acetic acid easily causes reactive oxygen species (ROS) accumulation, leading to programmed cell death (PCD). (4) Acetic acid also affects a membrane structure and inhibits intracellular translation.

Furan inhibitors mainly include furfural and hydroxymethylfurfural (HMF). Furfural is more toxic than HMF, and its concentration is generally 0 g/L to 5 g/L. HMF inhibits enzymes (such as alcohol dehydrogenase (ADH), aldehyde dehydrogenase (ALDH), and pyruvate dehydrogenase (PDH)) involved in metabolic processes, such as glycolysis and tricarboxylic acid (TCA) cycle, to reduce the conversion rates of glucose and xylose during fermentation. Saccharoinyces cerevisiae consumes a large number of coenzymes when converting furan inhibitors into corresponding alcohol compounds, which easily leads to the imbalance of intracellular redox and reduces the ability of cells to produce ATP. Furfural can induce the accumulation of ROS in Saccharomyces cerevisiae, trigger the PCD, and reduce ethanol yield.

Lignocellulose-derived phenol compounds are usually benzene ring-containing aromatic compounds, which are mainly produced from lignin formed during the degradation of lignin. Phenol inhibitors affect the selectivity and permeability of a cell membrane by disrupting the integrity of the cell membrane and the electrochemical gradient of the mitochondrial membrane. Therefore, improving the tolerance of Saccharomyces cerevisiae to inhibitors in a lignocellulose hydrolyzate is crucial for the development and application of the second-generation ethanol fuel.

The MAL33 gene participates in maltose metabolism and is a transcription factor that regulates the expression of the maltose permease gene MAL31 and maltase gene MAL32. It does not seem to be any reports or research on the function of MAL33 gene in improving the tolerance of Saccharomyces cerevisiae to H₂O₂ and the inhibitors in the lignocellulose hydrolyzates including acetic acid.

### SUMMARY

During the process of producing the second-generation ethanol fuel from a lignocellulose raw material in the prior art, Saccharomyces cerevisiae exhibits low tolerance to inhibitors in a lignocellulose hydrolyzate. In view of the above problem, the present disclosure provides an application of MAL33 gene deletion in improving the tolerance of Saccharomyces cerevisiae to inhibitors in a lignocellulose hydrolyzate.

The present disclosure adopts the following technical solutions:

An application of MAL33 gene deletion in improving the tolerance of Saccharomyces cerevisiae to inhibitors in a lignocellulose hydrolyzate is provided, where a MAL33 gene has a nucleotide sequence shown in SEQ ID NO: 1.

The MAL33 gene deletion of Saccharomyces cerevisiae may be achieved through the following steps: amplifying a MAL33 gene knockout fragment, transforming the MAL33 gene knockout fragment into Saccharomyces cerevisiae, and verifying a transformant.

The deletion of the MAL33 gene can improve the tolerance of Saccharomyces cerevisiae to acetic acid.

The deletion of the MAL33 gene can improve the tolerance of Saccharomyces cerevisiae to other typical inhibitors and H₂O₂ in the lignocellulose hydrolyzate.

The MAL33 gene-deleted Saccharomyces cerevisiae can exhibit improved tolerance to the mixed inhibitor in the lignocellulose hydrolyzate.

The other typical inhibitors may include vanillin, formic acid, HMF, or levulinic acid.

The present disclosure provides a MAL33 gene-deleted Saccharomyces cerevisiae strain, and experiments confirm that the tolerance of the MAL33 gene-deleted strain to acetic acid is greatly improved. The knockout of the MAL33 gene can also increase the tolerance of Saccharomyces cerevisiae to other inhibitors and H₂O₂. A lag period of the MAL33 gene-deleted Saccharomyces cerevisiae strain in a glucose and xylose medium (YPDX) with 3.5 g/L acetic acid is shortened by 24 h. A fermentation period of the Saccharomyces cerevisiae strain to produce ethanol through co-utilization of glucose and xylose is shortened by 20 h, which provides a basis for the construction of a Saccharomyces cerevisiae cell with high acetic acid resistance. The growth of the Saccharomyces cerevisiae strain in a glucose and xylose medium (YPDX) with a mixed inhibitor and the ethanol production of the Saccharomyces cerevisiae strain through the co-fermentation of glucose and xylose are superior to those of a control strain. The present disclosure provides an important method for improving the tolerance of Saccharomyces cerevisiae to inhibitors in a lignocellulose hydrolyzate and provides a theoretical basis for overcoming the technical bottleneck in the production of the second-generation ethanol fuel.

The present disclosure has the following technical effects:

(1) The present disclosure can improve the tolerance of Saccharomyces cerevisiae to acetic acid.

(2.) The present disclosure can improve the tolerance of Saccharomyces cerevisiae to other typical inhibitors and H₂O₂ in a lignocellulose hydrolyzate.

(3) The present disclosure can improve the growth of Saccharomyces cerevisiae in a mixed sugar medium YPDX with 3.5 g/L acetic acid, shorten the lag period by 24 h, and shorten the ethanol production cycle of co-fermentation of glucose and xylose by 20 h.

(4) The present disclosure can improve the growth of Saccharomyces cerevisiae in a mixed sugar medium YPDX with a mixed inhibitor in a lignocellulose hydrolyzate (including 0.68 g/L acetic acid, 0.23 g/L formic acid, 0.58 g/L levulinic acid, 0.48 g/L furfural, 0.63 g/L HMF, and 0.76 g/L vanillin) and the ethanol production of Saccharomyces cerevisiae through co-fermentation of glucose and xylose.

### BRIEF DESCRIPTION OF THE DRAWINGS

FIG. 1 shows the polymerase chain reaction (PCR) amplification results of an upstream homology arm and a downstream homology arm in the MAL33 gene in Example 1, where M: DL5000 DNA marker; 1: the upstream homology arm of the MAL33 gene obtained through PCR amplification with primers UPS-MAL33-F and UPS-MAL33-R; and 2: the downstream homology arm of the MAL33 gene obtained through PCR amplification with primers DOS-MAL33-F and DOS-MAL33-R.

FIG. 2 shows the PCR amplification results of a gene fragment of loxP-KanMX4-loxP in Example 1, where M: DL5000 DNA marker; and 1: the gene fragment of G418 resistance gene loxP-KanMX4-loxP obtained through PCR amplification with primers KanMX4-F and KanMX4-R.

FIG. 3 shows the PCR amplification results of a MAL33 gene knockout fragment in Example 1, where M: DL5000 DNA marker; and 1: the MAL133 gene knockout fragment obtained through fusion PCR with primers UPS-MAL33-F and DOS-MAL33-R.

FIG. 4 shows the PCR verification results of a transformant in Example 1, where M: DL5000 DNA marker; and 1: a PCR verification result obtained with genomic DNA (gDNA) of the transformant as a template and UPS-MAL33-F and DOS-MAL33-R as primers.

FIG. 5 shows the growth curves of the MAL33 gene-deleted strain BSPX051-3XI-mal33Δ and the control strain BSPX051-3XI in a YPD medium with 3 g/L acetic acid in Example 2.

FIG. 6 shows the gradient growth test results of the MAL33 gene-deleted strain BSPX051-3XI-mal33Δ and the control strain BSPX051-3XI on YPD plates with other inhibitors and H₂O₂ in Example 2.

FIG. 7 shows the growth curves of the MAL33 gene-deleted strain BSPX051-3XI-mal33Δ and the control strain BSPX051-3XI in a YPDX medium with 3.5 g/L acetic acid during oxygen-limited shake-flask fermentation in Example 2.

FIG. 8 shows the analysis results of glucose and xylose consumption and ethanol production during oxygen-limited shake-flask fermentation of the MAL33 gene-deleted strain BSPX051-3XI-mal33Δ and the control strain BSPX051-3XI in a YPDX medium with 3.5 g/L acetic acid in Example 2.

FIG. 9 shows the growth curves of the MAL33 gene-deleted strain BSPX051-3XI-mal33Δ and the control strain BSPX051-3XI in a YPDX medium with a mixed inhibitor during oxygen-limited shake-flask fermentation in Example 2.

FIG. 10 shows the analysis results of glucose and xylose consumption and ethanol production during oxygen-limited shake-flask fermentation of the MAL33 gene-deleted strain BSPX051-3XI-mal33Δ and the control strain BSPX051-3XI in a YPDX medium with a mixed inhibitor in Example 2.

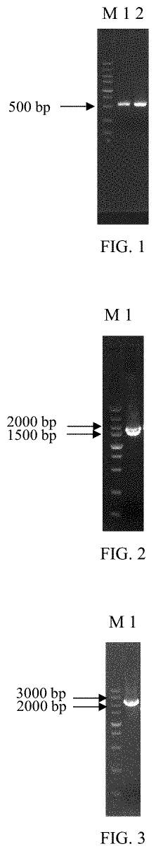

### DETAILED DESCRIPTION OF THE EMBODIMENTS

The content of the present disclosure will be described in detail below with reference to the specific accompanying drawings and examples. It should be noted that the above description is provided merely to explain the present disclosure, is a preferred implementation of the present disclosure, and is not intended to limit the present disclosure in any manner. Any simple modifications, equivalent changes, and modifications made to the above implementation according to the technical concept of the present disclosure should fall within the scope of the technical solution of the present disclosure.

Summary of media and experimental materials involved in the following examples:

1. Media

YPD medium: 20 g/L peptone, 10 g/L yeast powder, and 20 g/L glucose are mixed. When a solid medium is required, 20 g/L agar powder is added and the resulting mixture is sterilized at 115° C. for 30 min. When the YPD medium is used as a selection medium, G418 is added at a final concentration of 200 μg/mL. When the YPD medium is used as a fermentation medium, acetic acid is added at a final concentration of 3 g/L.

YPDX medium: 20 g/L peptone, 10 g/L yeast powder, 20 g/L glucose, and 20 g/L xylose are mixed and sterilized at 115° C. for 30 min. When the YPDX medium is used as a fermentation medium, acetic acid is added at a final concentration of 3.5 g/L.

2. Enzymes and Reagents

DNA polymerases used for PCR amplification are Phanta HS Super-Fidelity DNA Polymerase (P502-d1) and DL5000 DNA marker, which are purchased from Nanjing Vazyme Biotech Co., Ltd. Other materials and reagents are obtained from commercial sources unless otherwise specified.

### Example 1: Construction of a MAL33 Gene-Deleted Saccharomyces cerevisiae Strain

1. Extraction of Saccharomyces cerevisiae Genome

A Saccharomyces cerevisiae strain BY4741 was cultivated overnight in 5 mL of the YPD medium, resulting cells were collected, and gDNA of BY4741 was extracted.

2. Amplification of a Gene Knockout Fragment

(1) Amplification of upstream and downstream homology arms of MAL33: With the gDNA of the Saccharomyces cerevisiae BY4741 as a template, primers UPS-MAL33-F (5′-AATGGTCACTCCAAGTAACGGTATTGTGATTTCAACAGAA-3′, as shown in SEQ ID NO: 2) and UPS-MAL33-R (5′-TATTAAGGGTTGTCGACCTGATCTTGACAACTGAGCTCTTTCACAC-3′, as shown in SEQ ID NO: 3) (the underlined part was a homologous sequence upstream of a loxP-KanMX4-loxP fragment, which was for fusion PCR with loxP-KanMX4-loxP) and primers DOS-MAL33-F (5′-TGATATCAGATCCACTAGTGTAGGACCCTCATCACAATGATT-3′, as shown in SEQ ID NO: 4) (the underlined part was a homologous sequence downstream of loxP-KanMX4-loxP, which was for fusion PCR with loxP-KanMX4-loxP ) and DOS-MAL33-R (5′-TGAACTCAGAGAAATGGAATTGGGGTGCTA-3′, as shown in SEQ ID NO: 5) were used to conduct PCR amplification to obtain the upstream and downstream homology arms of the MAL33 gene that were each of about 500 bp and had a partial sequence of a G418 resistance gene loxP-KanMX4-loxP. PCR amplification was conducted under the following conditions: pre-denaturation at 95° C. for 10 min, denaturation at 95° C. for 15 s, annealing at 52° C. for 15 s, extension at 72° C. for 30 s, with 30 cycles, and final extension at 72° C. for 5 min.

(2) Amplification of the G418 resistance gene loxP-KanMX4-loxP: With a pUG6 plasmid as a template, primers KanMX4-F (5′-AGCTGAAGCTTCGTACGCTG-3′, as shown in SEQ ID NO: 6) and KanMX4-R (5′-GCATAGGCCACTAGTGGATCTG-3′, as shown in SEQ ID NO: 7) were used to conduct PCR amplification to obtain a gene fragment loxP-KanMX4-loxP with a loxP site that was of about 1,500 bp.

(3) Fusion PCR amplification of MAL33 upstream and downstream homology arms and loxP-KanMX4-loxP : With the upstream and downstream homology arms of the MAL33 gene and loxP-KanMX4-loxP as templates, primers UPS-MAL33-F and DOS-MAL33-R were used to conduct fusion PCR to obtain a MAL33 gene knockout fragment of about 2,600 bp. The PCR amplification was conducted under the following conditions: pre-denaturation at 95° C. for 10 min, denaturation at 95° C. for 15 s, annealing at 52° C. for 15 s, extension at 72° C. for 3 min and 50 s, with 30 cycles, and final extension at 72° C. for 5 min.

3. Transformation of the MAL33 Gene Knockout Fragment into Saccharemyces cerevisiae

The MAL33 gene knockout fragment amplified by the fusion PCR was transformed into Saccharomyces cerevisiae BSPX051-3XI by the lithium acetate transformation method, and screening was conducted on a YPD plate with 200 μg/mL G418 to preliminarily obtain a MAL33 gene-deleted transformant.

4. PCR verification of the transformant: Single colonies were picked from the screening plate and then cultivated at 30° C. and 200 rpm for 12 h to 24 h in a YPD liquid medium with 200 μg/mL G418, and gDNA of the transformant was extracted. With the gDNA as a template, primers UPS-MAL 33-F and DOS-MAL33-R were used to conduct PCR verification. The PCR amplification was conducted under the following conditions: pre-denaturation at 95° C. for 10 min, denaturation at 95° C. for 15 s, annealing at 52° C. for 15 s, extension at 72° C. for 3 min and 50 s, with 30 cycles, and final extension at 72° C. for 5 min. A band of about 2,600 bp was amplified by PCR, indicating that the MAL33 gene was successfully knocked out to obtain the MAL33 gene-deleted strain BSPX051-3XI-mal33Δ.

### Example 2: Test of the Tolerance of the MAL33 Gene-Deleted Strain to Acetic Acid, Other Typical Inhibitors, and H₂O₂

1. Evaluation of the Oxygen-Limited Shake-Flask Fermentation of the MAL33 Gene-Deleted Strain in a YPD Medium With 3 g/L Acetic Acid

Single colonies of each of the MAL33 gene-deleted strain BSPX051-3XI-mal33Δ and the control strain BSPX051-3XI were picked, inoculated into 5 mL of a YPD liquid medium, and subjected to a first activation in a shaker at 30° C. and 200 rpm for 12 h to 24 h until a turbid bacterial solution was obtained. The bacterial solution was transferred to 5 mL of a fresh medium and subjected to a second activation for 12 h to 24 h. An activated seed solution was inoculated into an oxygen-limited flask with 30 mL of a YPD+3 g/L acetic acid medium, the initial OD₆₀₀ was adjusted to 0.2, and the oxygen-limited shake-flask fermentation was conducted in a shaker at 30° C. and 200 rpm, where a sample was collected every few hours and the OD₆₀₀ of a fermentation broth was determined by a UV-Vis spectrophotometer. It can be seen from the results in FIG. 5 that the growth of BSPX051-3XI-mal33Δ is significantly better than the growth of BSPX051-3XI, indicating that the knockout of the MAL33 gene can improve the tolerance of Saccharomyces cerevisiae to acetic acid.

2. Evaluation of the Growth of the MAL33 Gene-Deleted Strain on a YPD Plate With Other Inhibitors in the Lignocellulose Hydrolyzate and H₂O₂

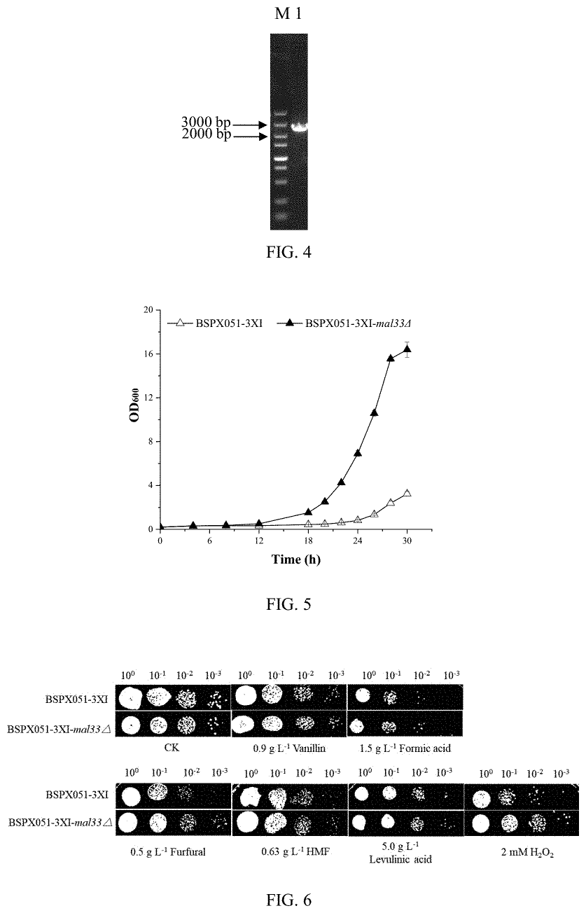

Single colonies of each of the MAL33 gene-deleted strain BSPX051-3XI-mal33Δ and the control strain BSPX051-3XI were picked, inoculated into YPD, and subjected to activation cultivation overnight at 30° C. under shaking. Cells in the later logarithmic growth phase were collected through centrifugation, washed with sterile water 3 times, suspended in 1 mL of sterile water, and cultivated in a 30° C. incubator for 9 h to consume endogenous nutrients, thereby facilitating the preparation of resting cells. A resting cell concentration was adjusted to obtain a cell suspension with OD₆₀₀ of about 1. The cell suspension was 10-fold diluted serially with three gradients (10⁰, 10⁻¹, 10⁻², and 10⁻³). 4 μL of a diluted solution was taken, added dropwise on a YPD plate with 3 g/L acetic acid, and cultivated at 30° C. for 2 d to 3 d. The growth of the colonies was observed and photographed, and results were stored. It can be seen from the results in FIG. 6 that the growth of BSPX051-3XI-mal33Δ on a YPD plate with other typical inhibitors in the lignocellulose hydrolyzate (such as vanillin, formic acid, HMF, and levulinic acid) is slightly better than the growth of the control strain BSPX051-3XI, indicating that the knockout of the MAL33 gene can also improve the tolerance of Saccharomyces cerevisiae to the other typical inhibitors in the lignocellulose hydrolyzate. It can also be seen from the results in FIG. 6 that the growth of BSPX051-3XI-mal33Δ on the plate with H2O₂ is better than the growth of the control strain, indicating that the knockout of the MAL33 gene can also improve the tolerance of Saccharomyces cerevisiae to H₂O₂.

Example 3: Evaluation of Oxygen-Limited Shake-Flask Fermentation of the MAL33 Gene-Deleted Strain in a Mixed Sugar Medium With 3.5 g/L Acetic Acid And a Mixed Sugar Medium With a Mixed Inhibitor

1. Evaluation of the Oxygen-Limited Shake-Flask Fermentation of the MAL33 Gene-Deleted Strain in a YPDX Medium With 3.5 g/L Acetic Acid

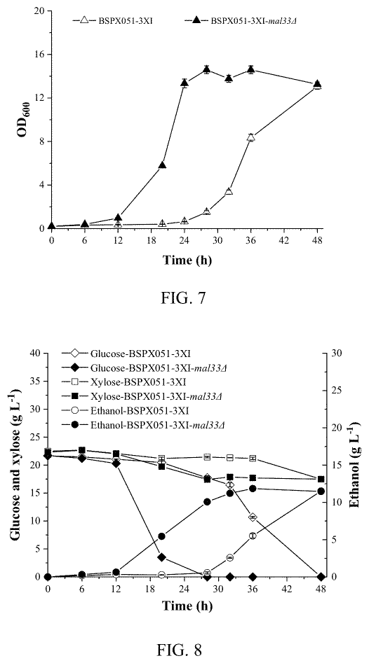

Single colonies of each of the MAL33 gene-deleted strain BSPX051-3XI-mal33Δ and the control strain BSPX051-3XI were selected, inoculated into 5 mL of a YPD liquid medium, and subjected to a first activation in a shaker at 30° C. and 200 rpm for 12 h to 24 h until a turbid bacterial solution was obtained. The bacterial solution was transferred to 5 mL of a fresh medium and subjected to a second activation for 12 h to 24 h. An activated seed solution was inoculated into 30 mL of a YPDX medium with 3.5 g/L acetic acid, the initial OD₆₀₀ was adjusted to 0.2, and the oxygen-limited shake-flask fermentation was conducted in a shaker at 30° C. and 200 rpm, where a sample was collected every few hours. The OD₆₀₀ of a fermentation broth was determined by a UV-Vis spectrophotometer, and the glucose and xylose consumption and ethanol production in a fermentation broth were analyzed by high-performance liquid chromatography (HPLC). It can be seen from the results in FIG. 7 that a fermentation lag period of BSPX051-3XI-mal33Δ in the YPDX medium with 3.5 g/L acetic acid is significantly shortened, and the growth of BSPX051-3XI-mal33Δ in the YPDX medium with 3.5 g/L acetic acid is significantly better than that of the control strain BSPX051-3XI. It can be seen from the HPLC analysis results in FIG. 8 that BSPX051-3XI-mal33Δ exhausts glucose within 28 h, while BSPX051-3XI exhausts glucose 48 h later. The xylose consumption and ethanol production capacity of BSPX051-3XI-mal33Δ are also significantly higher than the xylose consumption and ethanol production capacity of the control strain, indicating that the knockout of the MAL33 gene can improve the growth of Saccharomyces cerevisiae in the acetic acid-containing glucose and xylose medium and can also accelerate the ethanol production of Saccharomyces cerevisiae from glucose and xylose.

2. Evaluation of the Oxygen-Limited Shake-Flask Fermentation of the MAL33 Gene-Deleted Strain in a YPDX Medium With the Mixed Inhibitor in the Lignocellulose Hydrolyzate

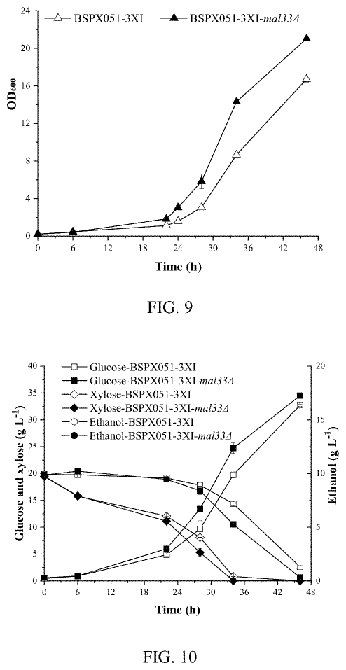

Single colonies of each of the MAL33 gene-deleted strain BSPX051-3XI-mal33Δ and the control strain BSPX051-3XI were selected, inoculated into 5 mL of a YPD liquid medium, and subjected to a first activation in a shaker at 30° C. and 200 rpm for 12 h to 24 h until a turbid bacterial solution was obtained. The bacterial solution was transferred to 5 mL of a fresh medium and subjected to a second activation for 12 h to 24 h. An activated seed solution was inoculated into 30 mL of a YPDX medium with a mixed inhibitor (including 0.68 g/L acetic acid, 0.23 g/L formic acid, 0.58 g/L levulinic acid, 0.48 g/L furfural, 0.63 g/L HMF, and 0.76 g/L vanillin), the initial OD₆₀₀ was adjusted to 0.2, and the oxygen-limited shake-flask fermentation was conducted in a shaker at 30° C. and 200 rpm, where a sample was collected every few hours. The OD₆₀₀ of a fermentation broth was determined by a UV-Vis spectrophotometer, and the glucose and xylose consumption and ethanol production in a fermentation broth were analyzed by HPLC. It can be seen from the results in FIG. 9 that the growth of BSPX051-3XI-mal33Δ in the YPDX medium with the mixed inhibitor is significantly better than that of the control strain BSPX051-3XI. It can be seen from the HPLC analysis results in FIG. 10 that the glucose and xylose consumption and ethanol production capacity of BSPX051-3XI-mal33Δ are also significantly higher than the glucose and xylose consumption and ethanol production capacity of the control strain, indicating that the knockout of the MAL33 gene can improve the growth of Saccharomyces cerevisiae in the medium with the mixed inhibitor in the lignocellulose hydrolyzate and can also accelerate the ethanol production through co-fermentation of glucose and xylose.

What is claimed:

1. An application of a MAL33 gene deletion in improving a tolerance of Saccharomyces cerevisiae to inhibitors in a lignocellulose hydrolyzate, wherein a MAL33 gene has a nucleotide sequence shown in SEQ ID NO: and the inhibitors comprise acetic acid, H₂O₂, vanillin, formic acid, 5-hydroxymethylfurfural (5-HMF), and levulinic acid.


SEQUENCE LISTING (7 sequences)

SEQ ID NO: 1 - DNA, 1407 bp, synthetic construct
  misc_feature 1..1407: note = The sequence is synthetized.
  source 1..1407: mol_type = other DNA
atgactttagtcaagtatgcatgcgactattgtcgtgtccgtcgagtaaagtgtgatggtaaaaaaccatgtagtcgctgtattgagcacaatttcgattgcacatatcagcaaccattaaaaaaaagaggctcaaagcctattggaactagaagcttaaagtatataccaaaggccaaaatgtttatagataataaaagttgtacggcagcggcagaaatattaatgaaggttcctaaaaaggtgattgatcagtgtttgaggctttatcacgacaatctatatgttatctggcctttactctcttatgatgaccttcacaagcttttggacgaagaatacaatgaccattacgtttattggtttctcgtagctctttcagcagccaatcttagcgatctacaaagcgaattagaatctgaggggggattttcgtttactggaaaacaattagccgttctctgtatgtcctcacgccaacaatttgatgaccttagcggcagagacattttccgaattatgacatattattgcttgctacgttgtttttcacagtcttctgatgtaagaaattcatacagactttgtcgtgaagctattggccttgttattgtagcagggttacatcgtgaaaaagcatacgaatcactatcatttcgtgagcaacagcttttacgcaaggtgtattatttgcttctcttgacggaaagatactattccgtatatgttcattgtgttacaagtttggataccacaatagctccacctcagccagagttcgtaacggaccctcgactttctttggatagcttttttgagatgattagagtatttactgtaccaggtaaatgcttttttgatgctttggctacggagtctactagcggttcttgcactgaagactcactgaaaaaaatatggaaagagcttcacacagcatcccttgaaatagaaccatggtcttatggctatgtggacatttcattttctcgacattggattagggcgctggcttggaagctagtgtttcagatgaatggtaccaagtttttctcaaacgccaataatgctcacatattggtcgaaattgcaaaggatatgctggacgacatattcttaactccaaacaacctgtatgatgtacatggtcctggaataccaatgaaatcattggaagtagccaatgcattggtagatatcgtaaataagtatgatcacaatatgaagttggaggcttggaatattttgtgcgatgtatccaagttcgttttctccctgaaacattgcaatcataaaatgtttcaaaggttttcaactaaatgtcagagtgctctaatcgatttgcctatctctagaccactgcgcctaaatgatgattccaaagatgaagacgacataattccttaa

SEQ ID NO: 2 - DNA, 40 bp, synthetic construct
  misc_feature 1..40: note = The sequence is synthetized.
  source 1..40: mol_type = other DNA
aatggtcactccaagtaacggtattgtgatttcaacagaa

SEQ ID NO: 3 - DNA, 46 bp, synthetic construct
  misc_feature 1..46: note = The sequence is synthetized.
  source 1..46: mol_type = other DNA
tattaagggttgtcgacctgatcttgacaactgagctctttcacac

SEQ ID NO: 4 - DNA, 42 bp, synthetic construct
  misc_feature 1..42: note = The sequence is synthetized.
  source 1..42: mol_type = other DNA
tgatatcagatccactagtgtaggaccctcatcacaatgatt

SEQ ID NO: 5 - DNA, 30 bp, synthetic construct
  misc_feature 1..30: note = The sequence is synthetized.
  source 1..30: mol_type = other DNA
tgaactcagagaaatggaattggggtgcta

SEQ ID NO: 6 - DNA, 20 bp, synthetic construct
  misc_feature 1..20: note = The sequence is synthetized.
  source 1..20: mol_type = other DNA
agctgaagcttcgtacgctg

SEQ ID NO: 7 - DNA, 22 bp, synthetic construct
  misc_feature 1..22: note = The sequence is synthetized.
  source 1..22: mol_type = other DNA
gcataggccactagtggatctg